(19) United States
(12) Patent Application Publication
(10) Pub. No.: US 2025/0284333 A1
(43) Pub. Date: Sep. 11, 2025

(54) PROGRAM, INFORMATION PROCESSING METHOD, AND INFORMATION PROCESSING APPARATUS

(71) Applicants: VIE, INC., Kamakura-shi, Kanagawa (JP); NTT DATA INSTITUTE OF MANAGEMENT CONSULTING, Inc., Tokyo (JP); TOWA PHARMACEUTICAL CO., LTD., Kadoma-shi, Osaka (JP)

(72) Inventors: Takuya IBARAKI, Chiyoda-ku, Tokyo (JP); Tomohiro KUSUTOMI, Kamakura-shi, Kanagawa (JP); Yasuhiko IMAMURA, Kamakura-shi, Kanagawa (JP)

(21) Appl. No.: 18/704,902

(22) PCT Filed: Oct. 25, 2022
(86) PCT No.: PCT/JP2022/039621

§ 371 Date: Oct. 17, 2024

Related U.S. Application Data
(63) Continuation of application No. PCT/JP2021/040131, filed on Oct. 29, 2021.

(30) Foreign Application Priority Data
Oct. 29, 2021 (WO) PCT/JP2021/040131

(51) Int. Cl. G06F 3/01; A61M 21/00; A61M 21/02

(52) U.S. Cl. CPC G06F 3/011; A61M 21/02; A61M 2021/0027; A61M 2021/005; G06F 2203/011

(57) ABSTRACT

A program causes a processor included in an information processing apparatus to perform: acquiring irritability information relating to irritability of a predetermined user, based on an electroencephalographic signal measured by an electroencephalographic measurement device worn by the predetermined user; determining whether or not the irritability information satisfies a predetermined condition relating to a degree of anger set for the predetermined user; selecting at least one content from a dataset that is associated with the predetermined user and that includes one or more contents when the predetermined condition is satisfied; and determining a calming effect of the content, based on difference information about the irritability information acquired during output of the selected content.

### TECHNICAL FIELD

The present invention relates to a program, an information processing method, and an information processing apparatus.

### BACKGROUND ART

Conventionally, there has been known a technique in which an emotion of a user is estimated based on an electroencephalographic signal and music that suits the emotion is reproduced, thereby controlling the emotion of the user and enabling the user to listen to music that is enjoyable for himself/herself (refer to, for example, Non-Patent Document 1).

### CITATION LIST

### Non-Patent Document

Non-Patent Document 1: Ehrlich S K, Agres K R, Guan C, Cheng G (2019), “A closed-loop, music-based brain-computer interface for emotion mediation”, [online], Mar. 18, 2019, PLOS ONE, [searched on Sep. 9, 2021], Internet <URL: https://doi.org/10.1371/journal.pone.0213516>

### SUMMARY

### Technical Problem

There is a possibility that the conventional technique does not allow a user, who has difficulty in regulating his or her own emotion for some reason, to generate or reproduce music which the user wants to listen to.

Meanwhile, for the elderly or dementia patients, behavioral and psychological symptoms of dementia (BPSD: psychological symptoms and behavioral abnormalities that appear secondary to core symptoms, such as hallucinations, delusions, depression, restlessness, excitement, and wandering), as well as decline in cognitive functions, are problematic. For example, irritability occurs at a high rate among dementia patients, which is a psychological and physical burden not only on the patients themselves but also on caregivers. In addition, music therapy for dementia has been studied for some time now, and it has been found that music therapy has a favorable effect on BPSD, in particular.

However, the type of music that elicits an effect of regulating the emotion differ from individual to individual, and thus, reproduction of appropriate music that suits each individual user to regulate the emotion of the user has not yet been achieved.

Therefore, it is an object of one aspect of the disclosed technique to provide a program, an information processing method, and an information processing apparatus that enable to appropriately select, in accordance with irritability of a user, a content that regulates the irritability of the user.

### Solution to Problem

According to one aspect of the disclosed technique, a program causes a processor included in an information processing apparatus to perform: acquiring irritability information relating to irritability of a predetermined user, based on an electroencephalographic signal measured by an electroencephalographic measurement device worn by the predetermined user; determining whether or not the irritability information satisfies a predetermined condition relating to a degree of anger set for the predetermined user; selecting at least one content from a dataset that is associated with the predetermined user and that includes one or more contents when the predetermined condition is satisfied; and determining a calming effect of the content, based on difference information about the irritability information acquired during output of the selected content.

According to one aspect of the disclosed technique, an information processing method includes: by a processor included in an information processing apparatus, acquiring irritability information relating to irritability of a predetermined user, based on an electroencephalographic signal measured by an electroencephalographic measurement device worn by the predetermined user; determining whether or not the irritability information satisfies a predetermined condition relating to a degree of anger set for the predetermined user; selecting at least one content from a dataset that is associated with the predetermined user and that includes one or more contents when the predetermined condition is satisfied; and determining a calming effect of the content, based on difference information about the irritability information acquired during output of the selected content.

According to one aspect of the disclosed technique, an information processing apparatus that includes a processor, wherein the processor performs: acquiring irritability information relating to irritability of a predetermined user, based on an electroencephalographic signal measured by an electroencephalographic measurement device worn by the predetermined user; determining whether or not the irritability information satisfies a predetermined condition relating to a degree of anger set for the predetermined user; selecting at least one content from a dataset that is associated with the predetermined user and that includes one or more contents when the predetermined condition is satisfied; and determining a calming effect of the content, based on difference information about the irritability information acquired during output of the selected content.

### Advantageous Effects of Invention

According to one aspect of the disclosed technique, a content that regulates irritability of a user in accordance with the irritability of the user can be appropriately selected.

### BRIEF DESCRIPTION OF DRAWINGS

FIG. 1 is a diagram illustrating an example of a system according to an Embodiment.

FIG. 2 is a diagram illustrating an example of an earphone set according to the Embodiment.

FIG. 3 is a diagram illustrating an example of a schematic cross section of an earphone according to the Embodiment.

FIG. 4 is a diagram illustrating an example of an anger level input screen used in Experiment A.

FIG. 5A is a diagram illustrating an example of the experimental result of Experiment B conducted on a subject A.

FIG. 5B is a diagram illustrating an example of the experimental result of Experiment B conducted on a subject B.

FIG. 6A is a diagram illustrating another example of the experimental result of Experiment B conducted on the subject A.

FIG. 6B is a diagram illustrating another example of the experimental result of Experiment B conducted on the subject B.

FIG. 7A is a diagram illustrating an example of the experimental results indicating the actual anger levels A of seven subjects in Experiment B.

FIG. 7B is a diagram illustrating an example of the experimental results indicating the estimated anger levels B of the seven subjects in Experiment B.

FIG. 8A is a diagram illustrating an example of the experimental results of Experiment C conducted on a subject C.

FIG. 8B is a diagram illustrating an example of the experimental results of Experiment C conducted on a subject D.

FIG. 9 is a block diagram illustrating an example of an information processing apparatus 30 according to an Example.

FIG. 10 is a block diagram illustrating an example of an information processing apparatus 50 according to the Example.

FIG. 11 is a flowchart illustrating an example of a model generation process performed by a server according to the Example.

FIG. 12 is a flowchart illustrating an example of a content output process according to the Example.

FIG. 13 is a flowchart illustrating an example of a dataset update process according to the Example.

FIG. 14 is a diagram illustrating an example of an application screen according to the Example.

### DESCRIPTION OF EMBODIMENTS

Hereinafter, Embodiments of the present invention will be described with reference to the drawings. The Embodiments described below are merely examples, and there is no intention to exclude various modifications and applications of techniques not explicitly described below. That is, the present invention can be implemented in various modifications without departing from the spirit of the invention. In the following description of the drawings, the same or similar parts are denoted by the same or similar reference numerals. The drawings are schematically illustrated and do not necessarily correspond to actual dimensions, ratios, and the like. The drawings may include portions having different dimensional relationships and ratios from one another.

### Embodiment

Hereinafter, an overview of a system according to an Embodiment will be described with reference to the drawings.

### <Overview of System>

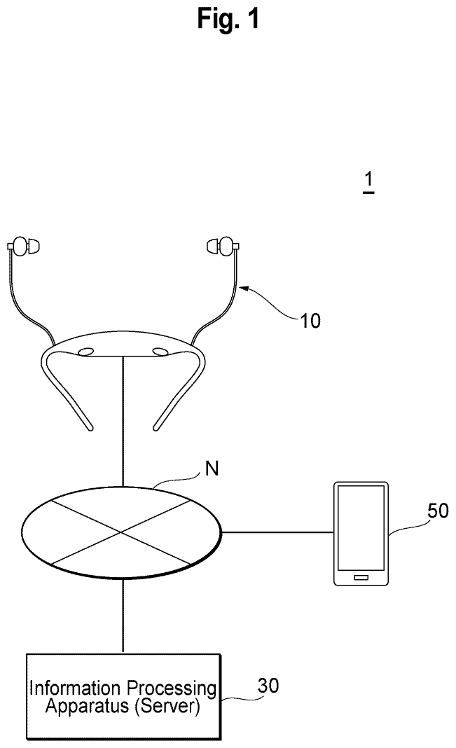

First, an example of an overview of a system 1 according to the Embodiment will be described with reference to FIG. 1. In the system 1, a user who is going to have an electroencephalogram measured wears, as an electroencephalographic measurement device, an earphone set 10 with which bio-electrodes are placed at the external auditory canals. While FIG. 1 illustrates a neckband-type earphone set 10 as an example, any type of earphones may be used as long as the earphone set can sense electroencephalographic signals from the external auditory canals. For example, an earphone set that acquires a reference signal from ear lobes, earphones that acquire a reference signal or a ground signal from other locations (other locations in the external auditory canals), completely wireless earphones, or the like can be used.

Alternatively, the user may wear, for example, a head gear that measures electroencephalograms by using the International 10-20 system as the electroencephalographic measurement device. Examples of the electroencephalographic measurement device include devices capable of measuring electroencephalograms such as a measuring device using scalp electrodes, a measuring device for measuring brain activity by using intracranial electrodes, a measuring device for measuring brain activity by using functional magnetic resonance imaging (fMRI), and a measuring device for measuring brain activity by using near-infrared spectroscopy (NIRS).

In the example illustrated in FIG. 1, the earphone set 10 acquires electroencephalographic signals from the external auditory canals and transmits the electroencephalographic signals to an information processing apparatus 30 or an information processing apparatus 50 via a network N. The earphone set 10 may perform predetermined processing on the electroencephalographic signals and transmit the processed electroencephalographic signals to the information processing apparatus 30 or the information processing apparatus 50. The predetermined processing includes amplification processing, sampling, filtering, difference calculation, and the like.

The information processing apparatus 30 is, for example, a server and calculates information indicating the emotion of the user from an electroencephalographic signal measured by the electroencephalographic measurement device. For example the information processing apparatus 30 may acquire emotion information indicating an emotion related to irritability by using a prediction model for extracting an emotion related to irritability from the electroencephalogram. The information processing apparatus 30 may improve the accuracy of the prediction model by using learning data annotated by the user. The information processing apparatus 30 may transmit the generated prediction model to the information processing apparatus 50.

The information processing apparatus 50 is, for example, a processing terminal such as a mobile terminal held by the user and sequentially acquires electroencephalographic signals from the earphone set 10. The information processing apparatus 50 feeds the sequentially acquired electroencephalographic signals to the prediction model to acquire emotion information indicating an emotion related to irritability.

When the emotion information satisfies a predetermined condition, the information processing apparatus 50 selects a content that is likely to regulate the emotion of the user and outputs the selected content to the earphone set 10. In the example illustrated in FIG. 1, the earphone set 10 is used as an example of the output device, and thus, the content includes sound. However, if an image output device is used as the output device, the content may include a moving image, a still image, animation, and the like. The information processing apparatus 50 has a function of a brain machine interface that operates a device based on the electroencephalographic signal so as to estimate the emotion of the user related to irritability from the electroencephalographic signal, perform processing for regulating the emotion, and operate the device.

Thus, the content that is likely to regulate the emotion of the user can be appropriately selected in accordance with the emotion of the user. For example, the content includes at least one of music, voice, sound data, a moving image, a photograph, an animation, a game, and the like. In the present disclosed technique, the content preferably includes sound, and more preferably, the content is music.

### <Configuration of Earphone Set>

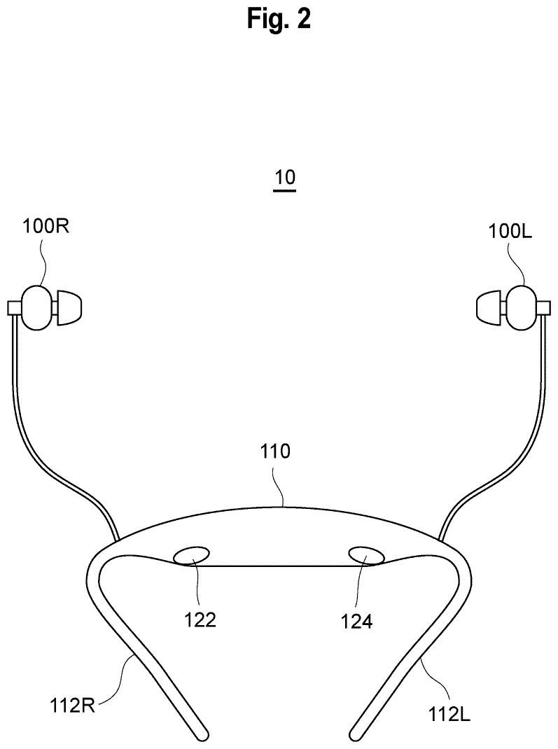

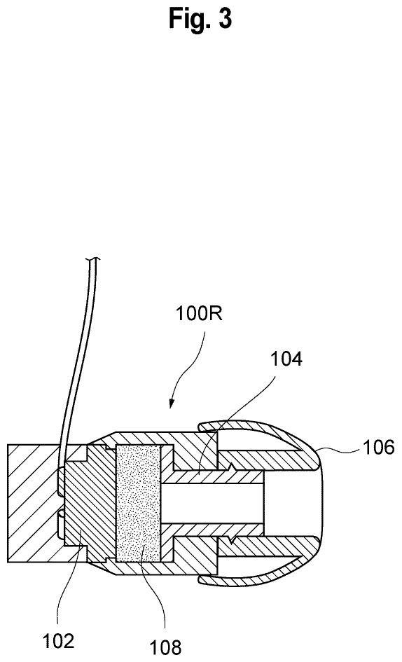

FIGS. 2 and 3 illustrate an overview of the earphone set 10 according to the Embodiment. The earphone set 10 is not limited to the examples illustrated in FIGS. 2 and 3, and any earphones can be applied to the technique of the present disclosure as long as the earphones can sense brain waves from the external auditory canals and can output the sensed brain waves to an external device.

FIG. 2 is a diagram illustrating an example of the earphone set 10 according to the Embodiment. The earphone set 10 illustrated in FIG. 2 includes a pair of earphones 100R and 100L and a neckband unit 110. Each of the earphones 100R and 100L is connected to the neckband unit 110 with a cable capable of signal communication. Alternatively, the earphones 100R and 100L may be connected by using wireless communication. Hereinafter, “R” and “L” will be omitted unless it is needed to distinguish between the right and left.

The neckband unit 110 includes a central member to be placed along the back of the neck and rod-like members (arms) 112R and 112L each of which has a shape curved along the corresponding side of the neck. Electrodes 122 and 124 for sensing an electroencephalographic signal are provided on the surface of the central member that comes into contact with the back side of the neck. The electrodes 122 and 124 are an electrode connected to the ground and a reference electrode. This arrangement makes it possible to position the electrodes 122 and 124 away from elastic electrodes provided on the ear tips of the earphones, as will be described below, so that the electroencephalographic signals can be accurately acquired. In addition, the neckband unit 110 may include a processing unit for processing the electroencephalographic signals and a communication device that communicates with the outside, or the processing unit and the communication unit may be provided in the earphones 100.

The rod-like members 112R and 112L on the respective sides of the neckband unit 110 are formed such that their tip end sides are heavier than their base sides (central member sides). Thus, the electrodes 122 and 124 are pressed against the neck of the wearer such that the electrodes 122 and 124 are properly in contact with the neck. For example, weights are provided on the respective tip end sides of the rod-like members 112R and 112L. Note that the positions of the electrodes 122 and 124 are not limited to those described above.

FIG. 3 is a diagram illustrating an example of a schematic cross section of the earphone 100R according to the Embodiment. The earphone 100R illustrated in FIG. 3 may be provided with an elastic member (for example, urethane) 108 between a speaker 102 and a nozzle 104, for example. By providing the elastic member 108, the vibration of the speaker 102 is prevented from transmitting to the elastic electrode of an ear tip 106 so that the elastic electrode of the ear tip 106 and the speaker 102 can be prevented from interfering with each other in terms of sound.

Further, although the ear tip 106 including the elastic electrode is located at the sound guide, the elasticity of the elastic electrode itself can prevent interference due to sound vibration. In addition, adopting the elastic member as the housing makes it difficult to transmit sound vibration to the elastic electrode of the ear tip 106, and interference due to sound vibration can be prevented.

The earphone 100 may include an audio sound processor and use the audio sound processor to cut a sound signal of a predetermined frequency (e.g., 50 Hz) or less, which corresponds to an electroencephalographic signal. In particular, although the audio sound processor cuts a sound signal of a frequency band of 30 Hz or less, which is likely to exhibit characteristics of the electroencephalographic signal, the audio sound processor may amplify a sound signal of frequencies around 70 Hz so as not to impair the bass sound.

This can prevent the sound signal and the electroencephalographic signal from interfering with each other. The audio sound processor may cut a sound signal of the predetermined frequency only when the electroencephalographic signal is being sensed.

The ear tip 106 conducts an electroencephalographic signal sensed from the external auditory canal to a contact point of the electrode provided in the nozzle 104. The electroencephalographic signal is transmitted from the ear tip 106 to a biometric sensor (not illustrated) inside the earphone 100 via the contact point. The biometric sensor outputs the sequentially-acquired electroencephalographic signals to a processing device provided in the neckband unit 110 via the cable or transmits the electroencephalographic signals to an external device. The ear tip 106 may be insulated from the housing including the biometric sensor or the audio sound processor.

### SUMMARY OF EXPERIMENTS

Next, experiments conducted by the inventors will be described. Through these experiments, generation of a prediction model for estimating irritability, validity of the estimated irritability, and relevance between the estimated irritability and music were examined.

### Experiment A

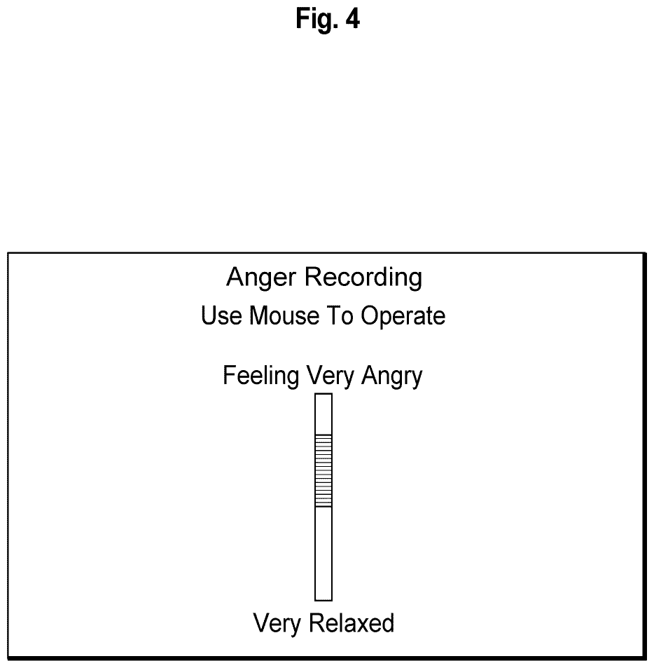

First, to record an electroencephalogram at a time when a subject feels anger, the following Experiment A was conducted on seven subjects.
The subject repeats a set of recalling an event of anger and relaxing about three times in the subject's own pace.
The subject uses an anger level gauge (for example, see FIG. 4) and enters the anger level or the degree of anger in real time by using a pointing device, for example, a mouse.
A wireless biometric device “Polymate” (registered trademark) Mini is used for recording an electroencephalogram. Three electrodes at both of the temporal parts (T7/8) and the top (near Cz) of the head are used to record the electroencephalographic signal at a 500 Hz sampling rate.

FIG. 4 is a diagram illustrating an example of an anger level input screen used in Experiment A. In Experiment A, electroencephalographic data on anger can be extracted by analyzing the actual electroencephalographic signal and the anger level entered by the subject. For example, a learning model for estimating anger is prepared, and the electroencephalographic data extracted by performing a predetermined process on the electroencephalographic signal and the anger level of the subject at the time are learned as learning data or training data. Thus, a prediction model for estimating anger is generated.

### Experiment B

Next, in Experiment B, the prediction model for anger estimation trained in Experiment A was used, and the seven subjects repeated the set of recalling anger and relaxing as in Experiment A.
The timing at which an anger level decoded from an electroencephalographic signal by using the prediction model has exceeded 2 standard deviations (SDs) within the immediately preceding 10 seconds is regarded as a “sign of anger”, and the first 15 seconds of one piece of music is randomly presented to the subject from among three to five pieces of music selected in advance by the subject him/herself as “music with which anger is likely to subside”.
The next piece of music is not reproduced until 5 seconds have elapsed from the end of the previous piece.
Similarly to Experiment A, the subject operates the gauge and enters his/her anger level in real time.

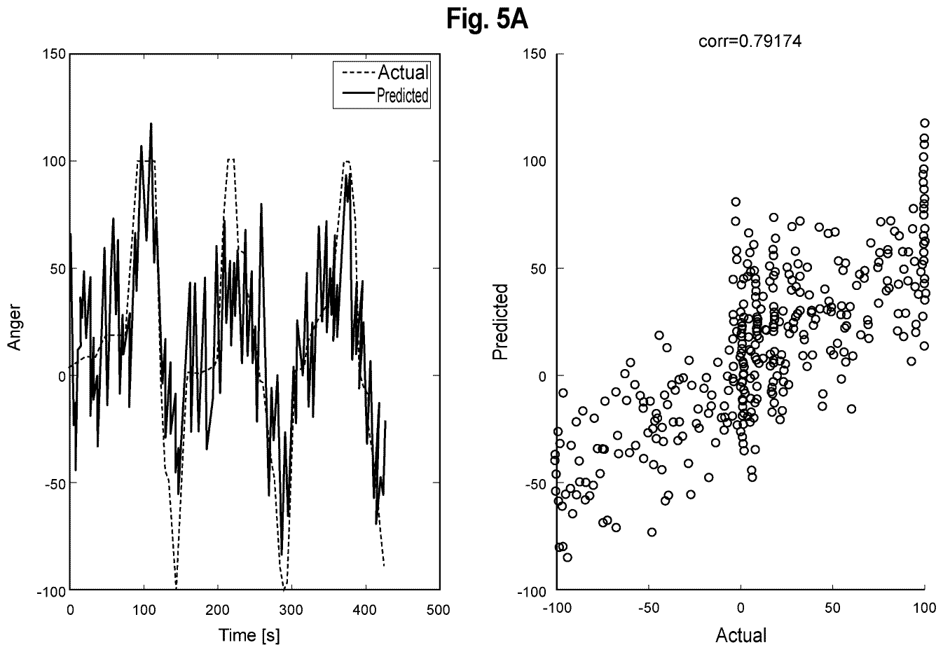

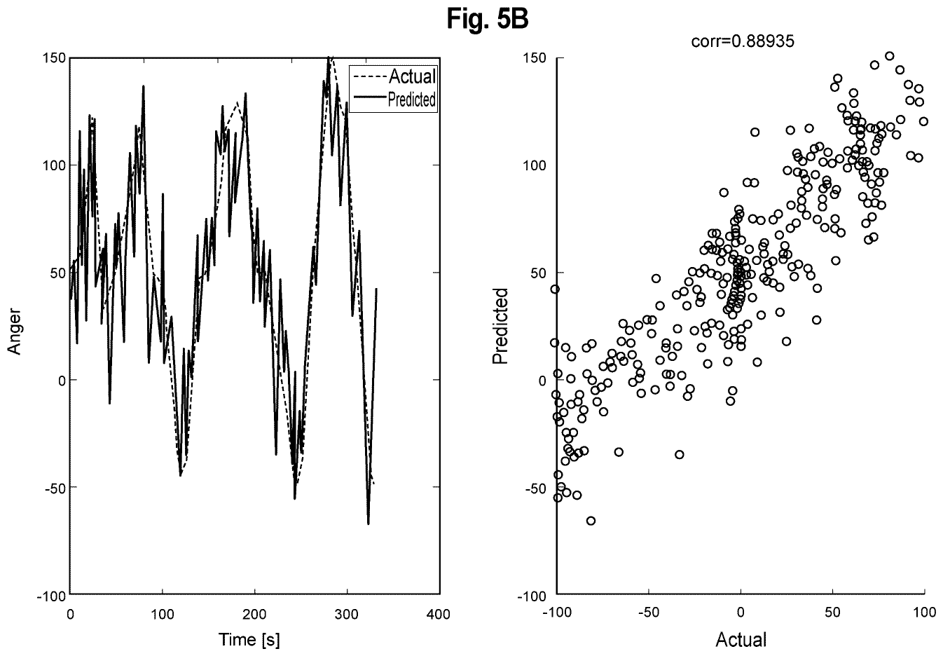

FIG. 5A is a diagram illustrating an example of the experimental result of Experiment B conducted on the subject A, and FIG. 5B is a diagram illustrating an example of the experimental result of Experiment B conducted on the subject B. In the graphs on the left side of FIGS. 5A and 5B, the horizontal axis represents time, and the vertical axis represents the value of anger level. The dotted line indicates an anger level A annotated by the subject, and the solid line indicates an anger level B estimated by using the prediction model. Hereinafter, the anger level A is also referred to as an actual anger level A, and the anger level B is also referred to as an estimated anger level B.

The graphs on the right side of FIGS. 5A and 5B are correlation graphs each illustrating the correlation between the actual anger level A and the estimated anger level B. As indicated by these correlation graphs, the correlation value between the actual anger level A and the estimated anger level B of the subject A is 0.79174, and the correlation value between the actual anger level A and the estimated anger level B of the subject B is 0.88935. Thus, the validity of the generated prediction model has been verified.

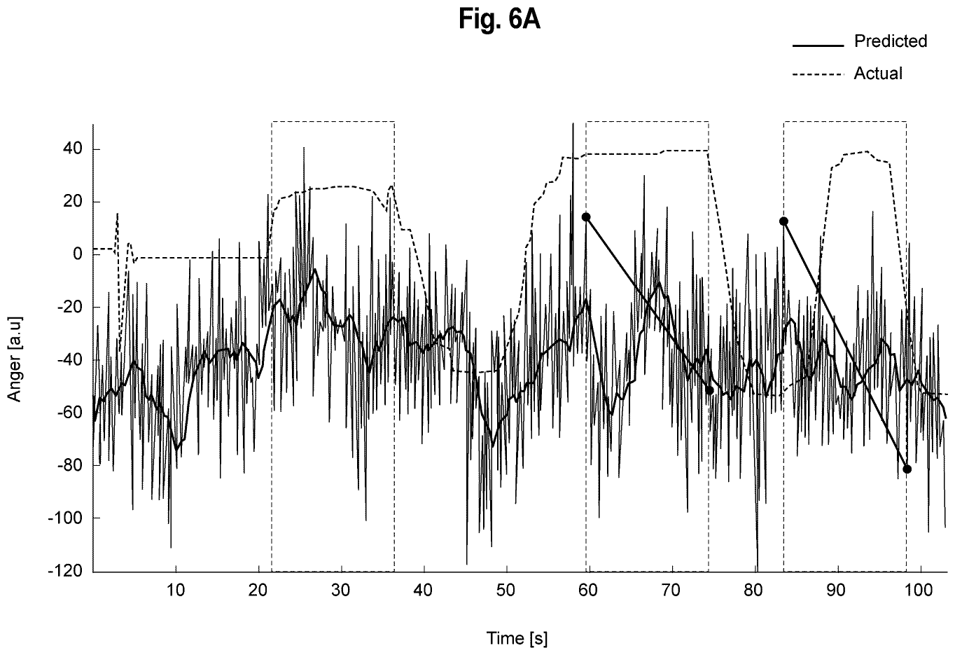

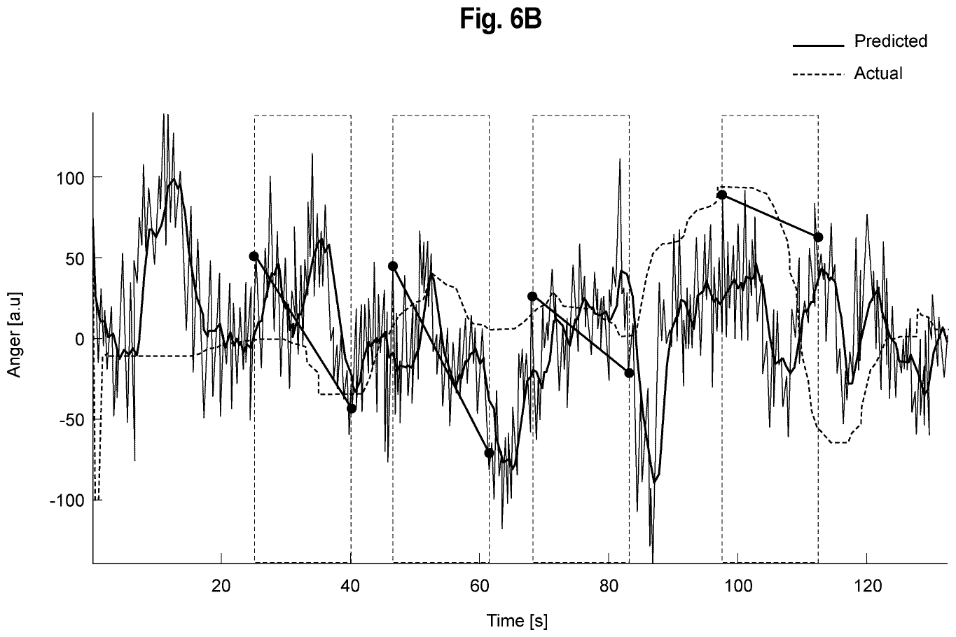

FIG. 6A is a diagram illustrating another example of the experimental result of Experiment B conducted on the subject A, and FIG. 6B is a diagram illustrating another example of the experimental result of Experiment B conducted on the subject B. The graphs illustrated in FIGS. 6A and 6B each indicate the relationship between the reproduction of music and the anger level. In the examples illustrated in FIGS. 6A and 6B, the dotted line indicates the actual anger level A, the solid line indicates the estimated anger level B, the thick line indicates the moving average of the estimated anger level B in a predetermined period, and the frame indicates a section (15 seconds) in which the music is reproduced.

In the examples illustrated in FIGS. 6A and 6B, for both the subjects A and B, the actual anger level A and the estimated anger level B have both decreased by listening to music. In addition, the line drawn within the section is a line connecting between a point representing the anger level B at the time when the reproduction of music has started and a point representing the anger level B at the time when the reproduction of music has finished. If the line within the section is inclined downward to the right, it can be said that the anger level has decreased.

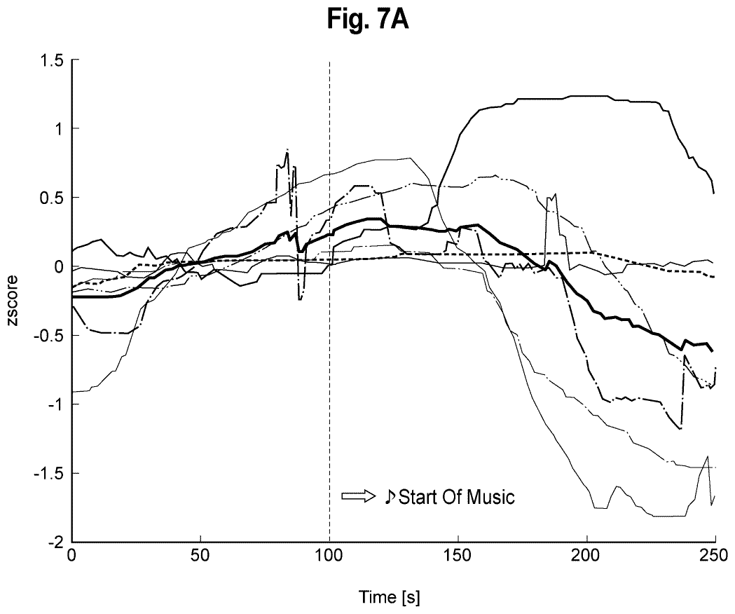

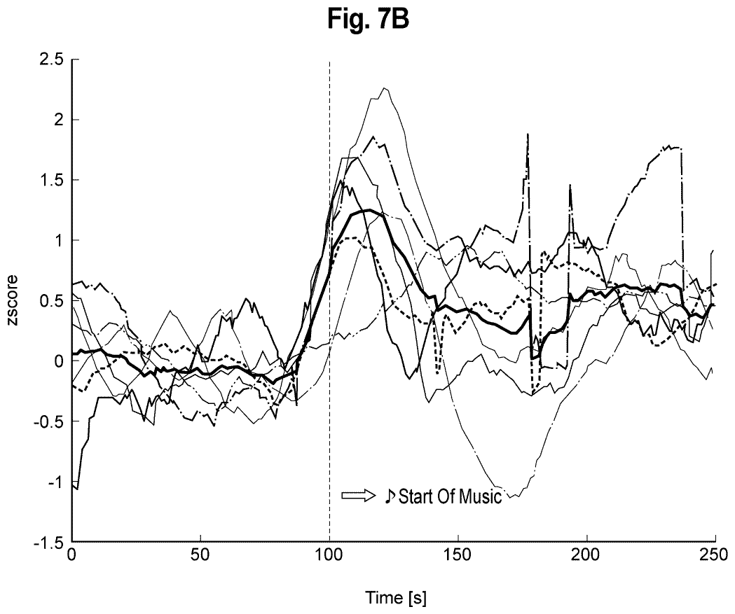

FIG. 7A is a diagram illustrating an example of the experimental results indicating the actual anger levels A of the seven subjects in Experiment B. In the example illustrated in FIG. 7A, although the anger levels of a few of the subjects have increased after the reproduction of music, the anger levels of the subjects on average have decreased after the reproduction of music.

FIG. 7B is a diagram illustrating an example of the experimental results indicating the estimated anger levels B of the seven subjects in Experiment B. In the example illustrated in FIG. 7B, anger has been detected by using the anger levels B before the reproduction of music, and then, the music has been reproduced. After the reproduction of music, the anger levels B have decreased.

The graphs illustrated in FIGS. 7A and 7B are created under the following conditions.
The estimated anger level B and the actual anger level A are standardized within the individual subject for each of the seven subjects.
The anger level A of 2.5 SDs or more is eliminated as noise.
The condition for starting music: when the anger level B has increased to 2 SDs or more in the data obtained within the immediately preceding 10 seconds
The mean of the data obtained in a period from 10 seconds before the start of the music to the start of the music is subtracted as a baseline from the anger level B at the start of the music, and the data on each individual before and after the start of the music is averaged.
Thick black line indicates the mean of the data of all the subjects.
A comparison is made between the data obtained within 2 seconds immediately after the start of the music and the data obtained within 2 seconds at the end of music (at 14 to 15 seconds after the start of the music) (paired t-test).

The result of the t-test obtained in the example illustrated in FIG. 7A was that the t-value=2.15 and the p-value=0.075, and the result of the t-test obtained in the example illustrated in FIG. 7B was that the t-value=2.69 and the p-value=0.036. Since the p-value obtained in the example illustrated in FIG. 7 is 0.05 or less, it can be said that there is a significant difference.

### Experiment C

Finally, in Experiment C, the prediction model for the anger estimation generated in Experiment A was used to examine whether or not a difference in regulating the anger level would be made depending on the presence or absence of music. Hereinafter, the conditions in Experiment C will be described.
(a) The condition for neurofeedback without music: the subject tries to suppress his/her anger level displayed on the monitor without listening to music.
(b) The condition for neurofeedback with music: the subject tries to find how to suppress his/her anger level through trial and error while playing various kinds of music. Under the above two conditions, two subjects try to lower their anger levels as much as possible.

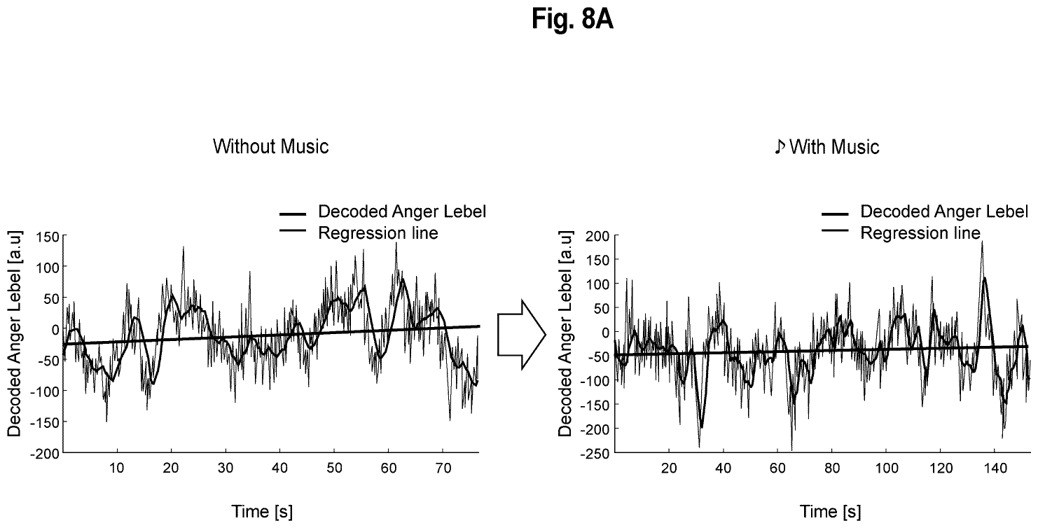

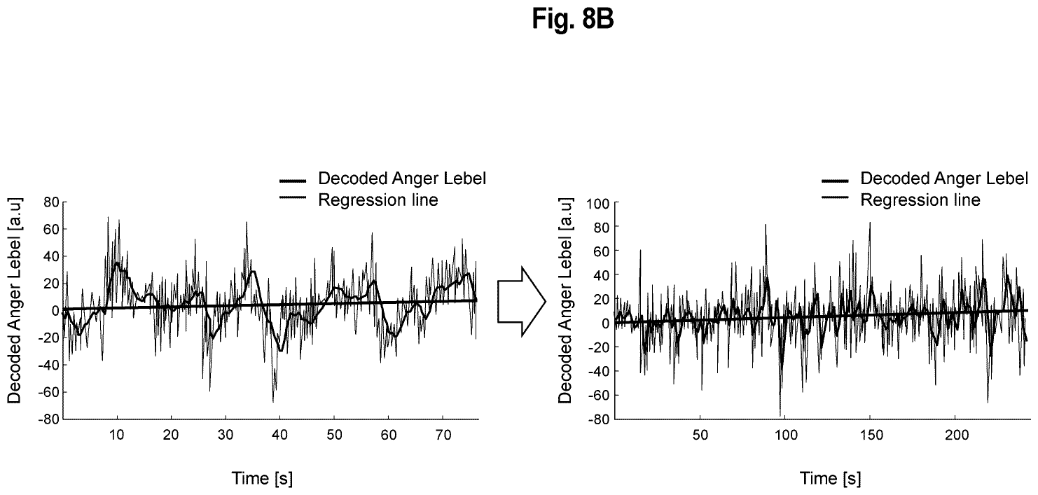

FIG. 8A is a diagram illustrating an example of the experimental result of Experiment C conducted on a subject C. FIG. 8B is a diagram illustrating an example of the experimental result of Experiment C conducted on a subject D. In each of FIGS. 8A and 8B, the graph on the left side indicates the experimental result without music, and the graph on the right side indicates the experimental result with music. The solid line indicates the estimated anger level B, the thick curved line indicates the moving average of the anger levels B in a predetermined period, and the thick straight line indicates the regression line of the moving average.
Inclination of regression line without music illustrated in FIG. 8A=0.37774
Inclination of regression line with music illustrated in FIG. 8A=0.12363
Inclination of regression line without music illustrated in FIG. 8B=0.08190
Inclination of regression line with music illustrated in FIG. 8B=0.04352

In the examples illustrated in FIGS. 8A and 8B, the estimated anger levels of the two subjects both tended to increase over time. However, as indicated by the inclinations of the regression lines in the graphs on the right side, it has been found that the neurofeedback using music tends to suppress the anger level to lower. For example, in the example illustrated in FIG. 8A, the case without music has an inclination of 0.37774, whereas the case with music has an inclination of 0.12363. That is, the inclination of the regression line with music is smaller. Thus, it can be said that the music has contributed to suppressing the anger level.

As described above, the following points have been verified from Experiments A to C.
The prediction model for estimating the anger level of an individual from the electroencephalographic signal was generated.
The anger level was estimated in real time.
Anger was detected from the estimated anger level, and the anger was calmed by the reproduction of music.

Hereinafter, an Example will be described. In this Example, irritability information about irritability is obtained from an electroencephalographic signal measured at the external auditory canals, the degree of anger of the user is determined based on the acquired irritability information, and music is reproduced if the degree of anger is high.

### Configuration Example of Server

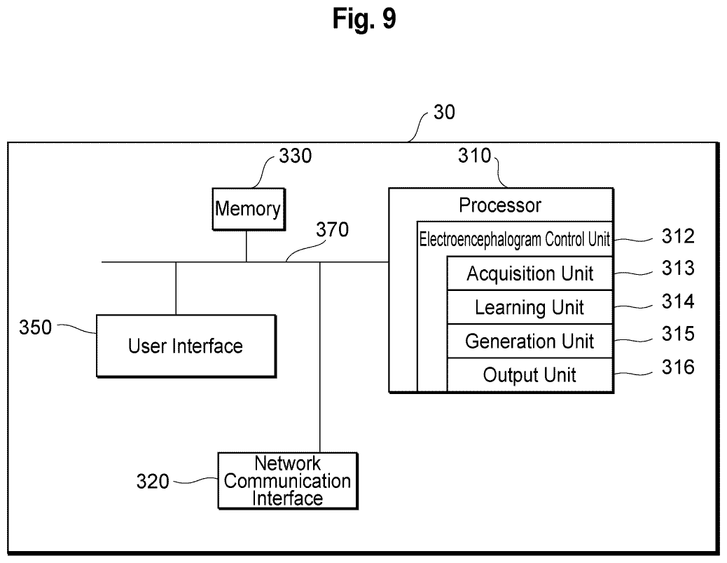

FIG. 9 is a block diagram illustrating an example of the information processing apparatus 30 according to the Example. The information processing apparatus 30 is, for example, a server and may be configured by one or more apparatuses. The information processing apparatus 30 processes electroencephalographic signals or electroencephalographic information and analyzes the emotion related to irritability of the user based on the electroencephalographic signal by using, for example, a learning function of an artificial intelligence. The information processing apparatus 30 is also referred to as a server 30. The information processing apparatus 30 is not necessarily a server and may be a general-purpose computer.

The server 30 includes one or more processors (CPUs: central processing units) 310, one or more network communication interfaces 320, a memory 330, a user interface 350, and one or more communication buses 370 for interconnecting these components.

The server 30 may optionally include, for example, the user interface 350 such as a display device (not illustrated) and a keyboard and/or a mouse (or an input device such as some kind of pointing device (not illustrated)).

The memory 330 is, for example, a high-speed random access memory such as a DRAM, an SRAM, a DDR RAM, or another random access solid storage device, and may also be a non-volatile memory such as one or more magnetic disk storage devices, optical disc storage devices, flash memory devices, or other non-volatile solid state storage devices. Further, the memory 330 may be a computer-readable non-transitory recording medium.

Another example of the memory 330 may be one or more storage devices remotely located from the processor 310. In one Example, the memory 330 stores the following programs, modules, and data structures, or a subset thereof.

The one more processors 310 read and execute the program from the memory 330 as needed. For example, the one or more processors 310 may include an electroencephalogram control unit 312, an acquisition unit 313, a learning unit 314, a generation unit 315, and an output unit 316 by executing the program stored in the memory 330. The electroencephalogram control unit 312 controls and processes sequentially acquired electroencephalographic signals and controls the following processing.

The acquisition unit 313 acquires electroencephalographic signals measured by the bio-electrodes included in the electroencephalographic measurement device, for example, the earphone set 10. The electroencephalographic measurement device is not limited to the earphone set 10.

The learning unit 314 holds a prediction model for estimating the degree of anger of the user by using irritability information based on the electroencephalographic signal. For example, the learning unit 314 performs supervised learning using, as training data, the irritability information in which the degree of anger has been annotated by the user.

The generation unit 315 acquires the learning result obtained through learning by the learning unit 314 and generates a prediction model that includes learned parameters and the like. The generation unit 315 may be a function included in the learning unit 314.

The output unit 316 outputs the prediction model generated by the generation unit 315 to a predetermined information processing apparatus 50 via the network communication interface 320.

As described above, the server 30 can generate a prediction model for estimating anger of the individual user who wears the electroencephalographic measurement device based on the electroencephalographic signal acquired at the external auditory canals. When the number of prediction models is equal to or more than a predetermined number, the generation unit 315 may classify the prediction models by feature of the irritability information and may associate the feature quantity of the irritability information with the classified prediction model. In this way, the prediction model does not need to be generated from the beginning for the user who can use the prediction model that has already been generated.

### Configuration Example of Processing Terminal

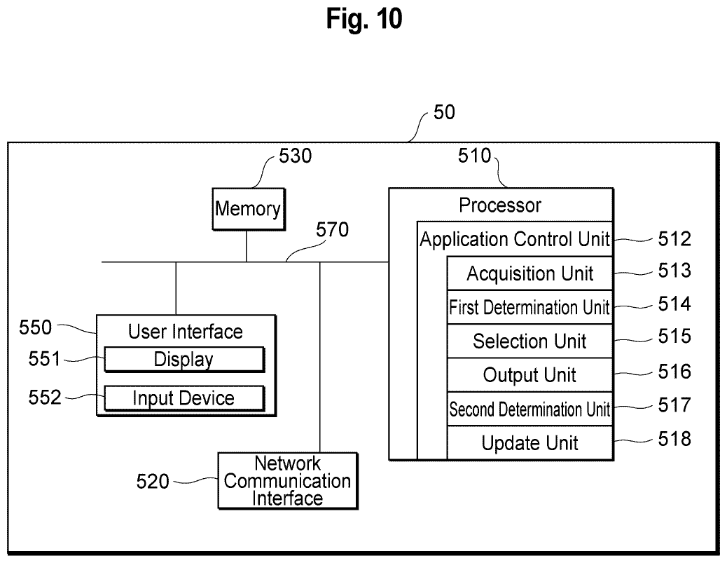

FIG. 10 is a block diagram illustrating an example of the information processing apparatus 50 according to the Example. The information processing apparatus 50 is, for example, a user terminal such as a mobile terminal (smartphone or the like), a computer, or a tablet terminal. The information processing apparatus 50 is also referred to as a processing terminal 50.

The processing terminal 50 includes one or more processors (e.g., CPUs) 510, one or more network communication interfaces 520, a memory 530, a user interface 550, and one or more communication buses 570 for interconnecting these components.

The user interface 550 includes a display device 551 and an input device (a keyboard and/or a mouse, some kind of pointing device, or the like) 552. The user interface 550 may be a touch panel.

The memory 530 is, for example, a high-speed random access memory such as a DRAM, an SRAM, a DDR RAM, or another random access solid storage device, and may also be a non-volatile memory such as one or more magnetic disk storage devices, optical disc storage devices, flash memory devices, or other non-volatile solid state storage devices. Further, the memory 530 may be a computer-readable non-transitory recording medium.

Another example of the memory 530 may be one or more storage devices remotely located from the processor 510. In one Example, the memory 530 stores the following programs, modules, and data structures, or a subset thereof.

The one or more processors 510 read and execute the program from the memory 530 as needed. For example, the one or more processors 510 may configure a control unit for controlling an application (hereinafter, also referred to as an “application control unit”) 512 by executing the program stored in the memory 530. The application control unit 512 is an application for processing electroencephalograms and includes, for example, an acquisition unit 513, a first determination unit 514, a selection unit 515, an output unit 516, a second determination unit 517, and an update unit 518.

The acquisition unit 513 acquires irritability information about irritability of a predetermined user based on the electroencephalographic signal measured by the electroencephalographic measurement device worn by the predetermined user. For example, the acquisition unit 513 may acquire the irritability information via the network communication interface 520 or may acquire the irritability information by performing predetermined processing on the electroencephalographic signal acquired via the network communication interface 520. The predetermined processing includes, for example, input processing in which the electroencephalographic signal is fed to the prediction model that has learned frequency conversion, filter processing, and the like for extracting the irritability information.

The first determination unit 514 determines whether or not the irritability information acquired by the acquisition unit 513 satisfies a predetermined condition relating to the degree of anger (anger level) set for the predetermined user. For example, the first determination unit 514 detects the sign of anger of the predetermined user by using a threshold provided for each user. The threshold is, for example, 2 standard deviations (2 SDs) of the irritability information about the predetermined user. Alternatively, an appropriate threshold for each user may be set through learning by the learning unit 314 of the information processing apparatus 30.

When the predetermined condition relating to the degree of anger is satisfied, the selection unit 515 selects at least one content from a dataset that is associated with the predetermined user and that includes one or more contents. For example, the dataset includes a content that has been selected in advance by the predetermined user or the family, relative, friend, or the like of the predetermined user. While the content is music as an example in the present Example, a content including sound or a content including an image may also be suitable.

In addition, when this application is used, the music having an effect of suppressing anger (calming effect) on the predetermined user may be set and accumulated in the dataset. In this case, the predetermined user or a user around the predetermined user may perform an operation of grasping the effect of the content while checking the irritability information about the predetermined user on a screen or the like and accumulating the content in the dataset. In this way, the contents in the dataset can appropriately be increased, and the dataset that does not bore the predetermined user can be created.

The application control unit 512 may monitor the irritability information about the predetermined user in real time during the reproduction of music and may automatically include the music being reproduced in the dataset if the music has an effect of suppressing the anger. This eliminates the need for the operation of accumulating the content in the dataset, and thus, the burden on the user can be reduced.

The output unit 516 outputs the content selected by the selection unit 515 to the earphone set 10 or a speaker provided in the information processing apparatus 50.

According to the above processing, the music that is likely to suppress the irritability of a predetermined user can be appropriately selected in accordance with the irritability of the predetermined user. For example, the irritability information is acquired based on the electroencephalogram of the predetermined user, the condition relating to the degree of anger that corresponds to the predetermined user is set, and music is selected from the dataset that corresponds to the predetermined user. In this way, customization for each individual user can be achieved.

The acquisition unit 513 may acquire irritability information sequentially output from the learning model when the electroencephalographic signals of the predetermined user are sequentially fed to the learning model, which has learned the irritability of the predetermined user by using the electroencephalographic signals of the predetermined user. For example, the learning model is the above-described prediction model for estimating anger. In this case, the acquisition unit 513 may feed the electroencephalographic signals measured by the earphone set 10 to the prediction model and acquire the irritability information output from this prediction model. The prediction model may be transmitted from the information processing apparatus 30 and held by the application control unit 512 or the acquisition unit 513. The acquisition unit 513 may acquire the irritability information output by earphone set 10 using the prediction model.

According to the above processing, it is possible to generate a prediction model for each user and use the irritability information about the individual user. In addition, a user-specific threshold or the like can be set for the irritability of the individual user so that customization for each individual user can be handled.

The learning model used for acquiring the irritability information may include a learning model trained by using supervised learning that has been annotated with irritability by the predetermined user. For example, as indicated in Experiment A, a more appropriate prediction model can be generated with the direct annotation by the user.

The predetermined condition used for determining irritability may include an anger determination condition set by using the irritability information about the predetermined user. For example, as illustrated in FIG. 7B, the level of the irritability information at its peak could vary depending on the user. Thus, the condition for detecting anger may be changed for each user, that is, for example, a threshold for the user A may be set based on the irritability information about the user A, and a threshold for the user B may be set based on the irritability information about the user B.

According to the above processing, it is possible to appropriately detect anger of each user by setting a condition for detecting anger by using the irritability information based on the actual electroencephalogram of the user. For example, as a condition for detecting anger, a threshold is set for the irritability information. The threshold may be set in advance for each user and may be appropriately changed by performing training on the prediction model as needed.

The selection unit 515 may change the content (e.g., music) being selected to another content based on the irritability information sequentially acquired by the acquisition unit 513. For example, if the irritability information is not suppressed or calmed during the output of the content, the selection unit 515 may select another content without outputting the currently selected content to the end. The condition for changing the output of the content includes, for example, that the mean irritability information during the output of the content is larger than the irritability information obtained at the start of the output of the content.

According to the above processing, the present system 1 can determine whether the selected content is appropriate based on the irritability information acquired in real time, and can change the selected content as needed. In addition, the selection unit 515 may give a lower priority to the content that has been determined that its suppressing effect or calming effect on the irritability information is low so that the content with a low priority is not to be selected.

The electroencephalographic measurement device is earphones having bio-electrodes for measuring electroencephalographic signals and may output at least one piece of music from the earphones. Examples of the earphones include the earphone set 10 as illustrated in FIG. 1, wireless earphones with bio-electrodes, and the like.

Since being the earphones, the electroencephalographic measurement device can serve as both the device for measuring the electroencephalographic signals and the device for outputting the selected content, and thus, it is possible to reduce the burden on the user in measuring electroencephalograms.

As described above, the information processing apparatus 50 can appropriately detect anger of the predetermined user by using the irritability information based on the electroencephalographic signal measured by the electroencephalographic measurement device, and can select and output the content that attempts to suppress the anger of the predetermined user. Further, the information processing apparatus 50 can acquire the irritability information about the predetermined user in real time and determine whether the content is appropriate. The determination of the appropriateness of the content being output will be described in detail below.

The second determination unit 517 determines the calming effect of the content being output based on difference information about the irritability information acquired during the output of the content selected by the selection unit 515. For example, the second determination unit 517 determines whether the irritability information indicates a shift toward sedation based on the difference information between the irritability information at the time of the output of the content and the irritability information after a predetermined time has elapsed from the output of the content. More specifically, if the difference information about the irritability information indicates that the irritability information has decreased from the irritability information at the time of the output of the content, the second determination unit 517 determines that the content being output has the calming effect. If the irritability information has not decreased even after the output of the content, the second determination unit 517 determines that the content being output has no calming effect.

The second determination unit 517 may calculate a mean value of the differential information about the irritability information within a predetermined time period from the start of the output of the content and determine the calming effect of the content being output based on the mean value. For example, if the mean value of the difference information (for example, inclination information) about the irritability information per unit time is equal to or less than a threshold, the second determination unit 517 may determine that the content being output has the calming effect. If the mean value is greater than the threshold, the second determination unit 517 may determine that the content being output has no calming effect.

According to the above processing, it is possible to determine whether the content being output actually has the calming effect on the irritability of the user. For example, by continuing to measure and analyze the electroencephalograms even while the content is being output, it is possible to determine, based on the electroencephalographic signals, the calming effect on the irritability.

The update unit 518 updates the dataset of the predetermined user based on the calming effect determined by the second determination unit 517. For example, the update unit 518 deletes the content having no calming effect from the dataset or lowers the priority of such a content. In addition, the update unit 518 may cause the selection unit 515 to preferentially select the content having the calming effect from the dataset by associating additional information, for example, a favorite mark, with the content having the calming effect.

According to the above processing, it is possible to update the dataset of the predetermined user in accordance with the actual calming effect of the content being output. As a result, the dataset can appropriately be customized for each user.

The second determination unit 517 may calculate the difference information by using the irritability information acquired within a predetermined time period from the start of the output of the content. The predetermined time period is, for example, 20 seconds after the output of the content.

According to the above processing, it is possible to determine whether or not the content has the calming effect within a predetermined time period from the start of the output of the content. This can improve the processing efficiency of the information processing apparatus 50. Further, it is possible to prevent the content that is unlikely to have the calming effect from continuing to be unnecessarily output.

The second determination unit 517 may subdivide the calming effect into a plurality of sets and determine, based on the magnitude of the difference information, one of the plurality of sets to which the difference information corresponds. For example, the second determination unit 517 sets a plurality of stepwise thresholds to the difference information, subdivides the calming effect, and associates the thresholds with the plurality of sets. For example, a first set is associated with “less than a first threshold”, a second set is associated with “equal to or more than the first threshold and less than a second threshold”, and a third set is associated with “equal to or more than the second threshold”. The first set indicates that the content produces no calming effect, the second set indicates that the content produces a calming effect, and the third set indicates that the content produces a significantly calming effect.

According to the above processing, it is possible to classify the calming effect of the content into levels by subdividing the calming effect based on the magnitude of the difference information.

The update unit 518 may classify the output content into the set corresponding to the magnitude of the difference information and update the dataset. For example, if the output content has a calming effect corresponding to the second set, the update unit 518 classifies the output content into the second set and updates the dataset, and if the output content has a calming effect corresponding to the third set, the update unit 518 classifies the output content into the third set and updates the dataset.

According to the above processing, the calming effect can be classified by level, and a set of corresponding contents can be generated for each level. Thus, the selection unit 515 is able to select a content corresponding to the level of the calming effect, for example.

The selection unit 515 may select any set in the dataset based on the difference information about the irritability information at a time when the predetermined condition indicating the anger of the predetermined user is satisfied. The selection unit 515 may select at least one content from among one or more contents classified into the selected set. For example, when the irritability information about the predetermined user indicates anger, if the differential information (e.g., inclination information) about the irritability information at a time when the anger has been determined is greater than a threshold (for example, if the anger threshold has suddenly been reached), the selection unit 515 selects a content from the third set. When the differential information about the irritability information at the time when the anger has been determined is smaller than the threshold, the selection unit 515 may select a content from the second set.

According to the above processing, when anger of the predetermined user is detected, it is possible to select an appropriate content to be output depending on whether the predetermined user has suddenly become angry or has slowly become angry. For example, when the predetermined user has suddenly become angry, a content can be selected from the set including the contents with a greater calming effect, and when the predetermined user has slowly become angry, a content can be selected from the set including the contents with a moderate calming effect.

The selection unit 515 may select at least one content from among one or more contents classified into the set other than the set including the output content such that the irritability information falls within a predetermined range of degrees of anger. For example, when the irritability information has rapidly decreased by selecting the content in the third set, the selection unit 515 switches the content being output by selecting the content of the second set so that the irritability information would not decrease excessively.

As a specific example, in a case where the predetermined range is indicated by an 11th threshold as the lower limit and a 12th threshold as the upper limit, when the differential information about the irritability information passes the threshold and becomes equal to or less than the 12th threshold after the content in the third set has been output, the selection unit 515 selects the content in the second set. When the irritability information becomes equal to or less than the eleventh threshold, the content may be switched from the content of the third set to the content of the second set. When the irritability information greater than the 12th threshold continues for a predetermined time during the output of the content of the second set, the selection unit 515 may select the content in the third set.

According to the above processing, it is possible to cause the irritability to fall within the appropriate predetermined range by switching the content in view of the calming effect of the content.

### Operations

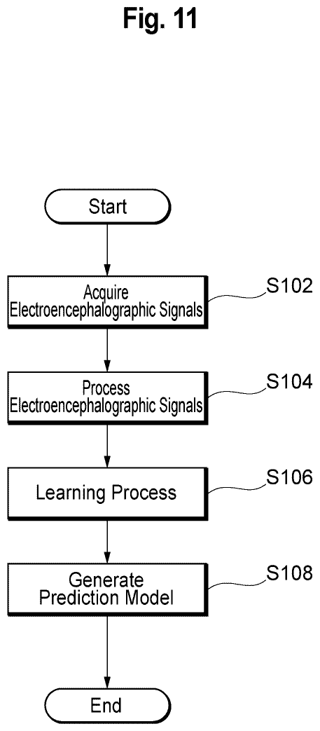

Next, the operations of the system 1 according to the Example will be described. FIG. 11 is a flowchart illustrating an example of a model generation process performed by the server 30 according to the Example. In the example illustrated in FIG. 11, a process in which the server 30 generates a learning model will be described.

In step S102, the acquisition unit 313 acquires electroencephalogram information measured by bio-electrodes each of which is in contact with the external auditory canal of the predetermined user. For example, the acquisition unit 313 acquires electroencephalogram information measured by the bio-electrodes provided on the ear tips of the earphones.

In step S104, the acquisition unit 313 performs preprocessing (processing treatment) for acquiring irritability information on the acquired electroencephalographic signals. The processing treatment includes at least one of sampling processing, filtering processing, frequency conversion processing, addition, subtraction, multiplication, and division processing, and the like.

In step S106, the learning unit 314 performs learning by using training data including the irritability information based on the electroencephalogram information and the result of annotation performed by the predetermined user. For example, the learning unit 314 uses a prediction model for estimating anger. When electroencephalogram information is fed to the prediction model, the prediction model outputs irritability information.

In step S108, the generation unit 315 generates a prediction model that has learned the irritability information extracted from the electroencephalographic signals. For example, when the irritability information based on the electroencephalogram information has been learned by the learning unit 314, the generation unit 315 generates a prediction model that predicts the irritability of the predetermined user from the electroencephalogram information. The prediction model may output the irritability information including the result of detecting the sign of anger from the electroencephalographic signals.

According to the above process, based on the electroencephalographic signals acquired in the external auditory canals, a prediction model for anger estimation can be generated for each individual user who wears the electroencephalographic measurement device.

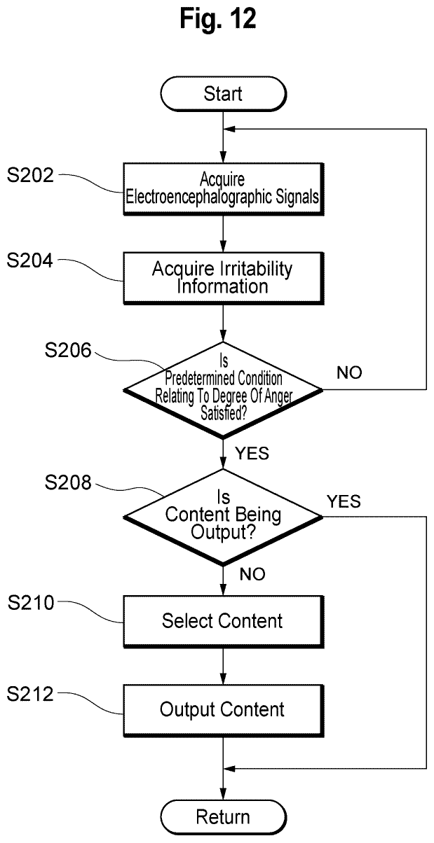

FIG. 12 is a flowchart illustrating an example of a content output process according to the Example. In the example illustrated in FIG. 12, the process will be described as a process performed by the information processing apparatus 50. However, the process may be performed by the earphone set 10.

In step S202, the acquisition unit 513 sequentially acquires electroencephalographic signals from the earphone set 10 via the network communication interface 520.

In step S204, the acquisition unit 513 sequentially acquires the irritability information by performing predetermined processing on the electroencephalographic signals. In the case where the earphone set 10 processes the electroencephalographic signals to calculate the irritability information, the acquisition unit 513 sequentially acquires the irritability information from the earphone set 10.

In step S206, the determination unit 514 determines whether or not the sequentially acquired irritability information satisfies a predetermined condition relating to the degree of anger. If the predetermined condition relating to the degree of anger is satisfied (YES in step S206), the processing proceeds to step S208. If the processing relating to the degree of anger is not satisfied (NO in step S206), the processing returns to step S202.

In step S208, the determination unit 514 determines whether or not the content is being output. If the content is not being output (NO in step S208), the processing proceeds to step S210, and if the content is being output (YES in step S208), the processing returns to step S202. The order of the determination processing in steps S206 and S208 may be switched. Even while the content is being output, the processing may proceed to step S210 if the predetermined time period or the predetermined condition relating to the degree of anger is satisfied.

In step S210, the selection unit 515 selects at least one content from the dataset that is associated with the predetermined user and that includes one or more contents.

In step S212, the output unit 516 outputs the selected content to the earphone set 10, the speaker of the information processing apparatus 50, or the speaker of another device.

According to the above process, it is possible to appropriately detect anger of the predetermined user by using the irritability information based on the electroencephalographic signal measured by the electroencephalographic measurement device, and to select and output the content for attempting to suppress the anger of the predetermined user.

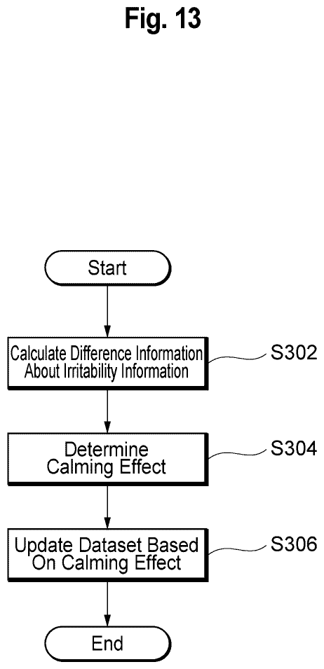

FIG. 13 is a flowchart illustrating an example of a dataset update process according to the Example. The example illustrated in FIG. 13 includes processing performed while the content selected by the selection unit 515 is being output to the user.

In step S302, the second determination unit 517 calculates difference information about the irritability information acquired during the output of the content selected by the selection unit 515. The difference information includes, for example, a differential value (inclination) per unit time.

In step S304, the second determination unit 517 determines the calming effect of the content being output based on the difference information. For example, the second determination unit 517 determines the calming effect based on the difference information between the irritability information at the time of the output of the content and the irritability information after a predetermined time has elapsed from the output of the content. More specifically, if the difference information about the irritability information indicates that the irritability information has decreased by a predetermined value or more, compared to the irritability information at the time of the output of the content, the second determination unit 517 may determine that the content being output has the calming effect. If the irritability information has not decreased after the output of the content, the second determination unit 517 may determine that the content being output has no calming effect.

In step S306, the update unit 518 updates the dataset of the predetermined user based on the calming effect determined by the second determination unit 517. For example, the update unit 518 deletes the content having no calming effect from the dataset or lowers the priority of such a content. In addition, the update unit 518 may cause the selection unit 515 to preferentially select the content having the calming effect from the dataset by specifying such a content.

According to the above process, it is possible to update the dataset of the predetermined user based on the actual calming effect of the content being output. As a result, the dataset can be appropriately customized for each user.

### Example of Screen

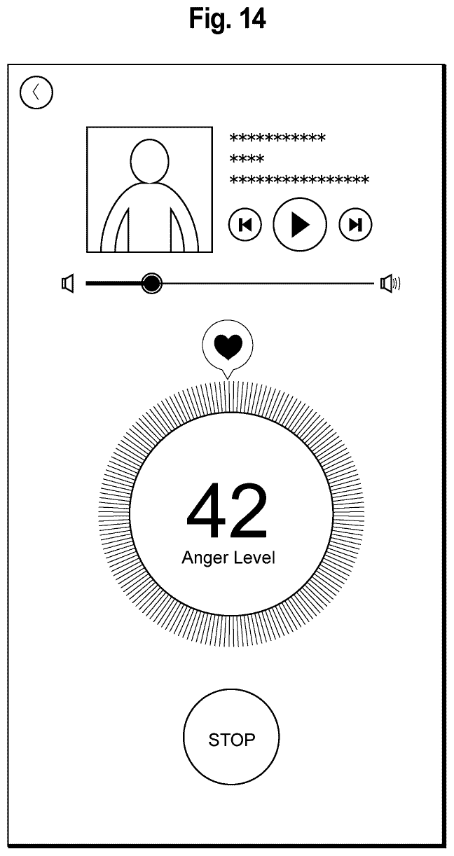

FIG. 14 is a diagram illustrating an example of an application screen according to the Example. In the example illustrated in FIG. 14, the irritability information based on the electroencephalographic signal is displayed in real time during the reproduction of music, which is an example of the content. For example, in the upper portion of the screen, information about the selected music is displayed, and operations such as play, stop, fast forward, rewind, etc. can be performed on the music.

In the center of the screen, a score value indicating real-time irritability information is displayed and sequentially updated. For example, a larger score value indicates a higher anger level, and a smaller score value indicates a calmer state. In the example illustrated in FIG. 14, the score value of the irritability information is denoted as “Anger Level”.

When this “Anger Level” starts to decrease during the reproduction of music, it can be determined that this music is effective for irritability. Therefore, the user using the information processing apparatus 50 may, for example, press a heart-mark symbol to include the music being reproduced in the playlist of the user who is the electroencephalographic measurement target. Note that, when the “Anger level” has decreased to a predetermined threshold or less during the reproduction of music, the selection unit 515 may automatically register the music being reproduced in the playlist as described above. The “STOP” button is a button for stopping the measurement of the electroencephalogram, and when the “STOP” button is pressed, the application control unit 512 may transmit a command for stopping the measurement of the electroencephalogram to the earphone set 10.

In the screen example illustrated in FIG. 14, a function of changing the music being reproduced may be added, and the display of the irritability information may be changed such that the irritability information is displayed as time-series data in a graph. The user using the information processing apparatus 50 can grasp the irritability of the user, who is the electroencephalographic measurement target, in real time. The user using the information processing apparatus 50 and the user who is the electroencephalographic measurement target may be the same user or may be different users.

### Examples of Application

The above-described application can be suitably applied to a care facility or the like that has users having a cognitive disorder. For example, the user having a cognitive disorder wears the electroencephalographic measurement device, and a caregiver or a family member uses the information processing apparatus 50. The user having a cognitive disorder frequently becomes angry. Therefore, by detecting the timing when the user becomes angry, the content having a calming effect on this user can be output. In this way, efforts can be made to prevent the user, who is the electroencephalographic measurement target, from becoming angry before that happens. For example, although music having a calming effect on anger varies for each user, if the music having a calming effect is registered as a playlist, music can be selected and output from the playlist prepared for each user.

For example, a family member of the user having a cognitive disorder can select music pieces for the playlist, or as described above, the user using the information processing apparatus 50 can determine the presence or absence of the effect of a music piece being reproduced while viewing the real-time irritability information and register the music piece in the playlist based on the determination. This is based on the known finding that music therapy is effective for the irritability of dementia patients and effective music varies for each user.

While music has been used as the content in the above examples, the content may be a moving image including a sound element, a game, or the like. Further, instead of using music, a still image, a moving image not including a sound element, an animation, or the like may be tested in the above-described experiments A and B, for example, and the effective content may be specified for each user.

### Modifications

The Embodiment and Example described above are merely examples for describing the technique of the present disclosure and are not intended to limit the technique of the present disclosure thereto, and various modifications can be made to the technique of the present disclosure without departing from the gist thereof.

### REFERENCE SIGNS LIST

1 System
10 Earphone set
30, 50 Information processing apparatus
100 Earphones
104 Nozzle
106 Ear tip (elastic electrode)
310 Processor
312 Electroencephalogram control unit
313 Acquisition unit
314 Learning unit
315 Generation unit
316 Output unit
330 Memory
310 Processor
512 Application control unit
513 Acquisition unit
514 First determination unit
515 Selection unit
516 Output unit
517 Second determination unit
518 Update unit
530 Memory

What is claimed is:

1. An information processing method, comprising:
by a processor included in an information processing apparatus,
acquiring irritability information relating to irritability of a predetermined user, based on an electroencephalographic signal measured by an electroencephalographic measurement device worn by the predetermined user;
determining whether or not the irritability information satisfies a predetermined condition relating to a degree of anger set for the predetermined user;
selecting at least one content from a dataset that is associated with the predetermined user and that includes one or more contents when the predetermined condition is satisfied; and
determining a calming effect of the content, based on difference information about the irritability information acquired during output of the selected content.

2. The information processing method according to claim 1, further comprising updating the dataset, based on the determined calming effect.

3. The information processing method according to claim 1, wherein
the determining includes calculating the difference information by using the irritability information acquired within a predetermined time period from a start of the output of the content.

4. The information processing method according to claim 2, wherein
the determining include, with the calming effect being subdivided into a plurality of sets, determining, based on magnitude of the difference information, one of the plurality of sets to which the difference information corresponds.

5. The information processing method according to claim 4, further comprising classifying an output content into the corresponding set and updating the dataset.

6. The information processing method according to claim 5, wherein
the selecting includes:
selecting any set included in the dataset, based on difference information about the irritability information at a time at which the predetermined condition is satisfied; and
selecting at least one content from among one or more contents classified into the selected set.

7. The information processing method according to claim 5, wherein
the selecting includes selecting at least one content from among one or more contents classified into a set other than a set including the output content so that the irritability information falls within a predetermined range of degrees of anger.

8. A program causing a processor included in an information processing apparatus to perform:
acquiring irritability information relating to irritability of a predetermined user based on an electroencephalographic signal measured by an electroencephalographic measurement device worn by the predetermined user;
determining whether or not the irritability information satisfies a predetermined condition relating to a degree of anger set for the predetermined user;
selecting at least one content from a dataset that is associated with the predetermined user and that includes one or more contents when the predetermined condition is satisfied; and
determining a calming effect of the content, based on difference information about the irritability information acquired during output of the selected content.

9. An information processing apparatus that includes a processor, wherein
the processor performs:
acquiring irritability information relating to irritability of a predetermined user, based on an electroencephalographic signal measured by an electroencephalographic measurement device worn by the predetermined user;
determining whether or not the irritability information satisfies a predetermined condition relating to a degree of anger set for the predetermined user;
selecting at least one content from a dataset that is associated with the predetermined user and that includes one or more contents when the predetermined condition is satisfied; and
determining a calming effect of the content, based on difference information about the irritability information acquired during output of the selected content.
